(19) United States
(12) Patent Application Publication
(10) Pub. No.: US 2007/0042498 A1
(43) Pub. Date: Feb. 22, 2007

(54) Method for assaying tropolone comprising complexing this molecule with cu (II)

(76) Inventor: Martin Ebner, Oxford (GB)

Correspondence Address: NIXON & VANDERHYE, PC, 901 NORTH GLEBE ROAD, 11TH FLOOR, ARLINGTON, VA, 22203, US

(21) Appl. No.: 10/568,497

(22) PCT Filed: Aug. 11, 2004
(86) PCT No.: PCT/EP04/08985

§ 371 Date: Feb. 16, 2006

(30) Foreign Application Priority Data
Aug. 19, 2003 (GB) 0319451.1

(51) Int. Cl. G01N 33/50

(52) U.S. Cl. 436/128

(57) ABSTRACT

A method for recovering and analyzing trace amounts of Tropolone from biological samples is devised.

The present invention relates to the field of analytical chemistry and production of recombinant protein from cell culture. It devises a method for determining analytical amounts of tropolone, which is a cell culture supplement for in animal cell culture and may contaminate product protein purified from such cell culture supernatant.

Tropolone, 2-hydroxy-2,4,6-cycloheptatrien-1-one, is a well-known iron chelator that is supplemented to protein-free animal cell culture medium to allow of iron uptake by the cells, as described in EP-610 474 B1. Tropolone acts in this sense much like natural siderophores known from bacteria. It excels both by its suitably reversible iron chelating properties and its non-toxicity, that is it interferes not with any cellular functions and does not negatively affect growth of animal cells in the relevant dosing.

As a synthetic chemical, any biopharmaceutical product protein harvested from tropolone-supplemented cell culture needs to be tested for trace amounts contamination by carry over of tropolone in the course of purification. A both sensitive and robust analytical method allowing of sufficient resolution of tropolone is required.

It is an object of the present invention to devise an analytical method for assaying tropolone from cell culture supernatant or proteinaceous solution, in particular from proteinaceous solution comprising a product protein that is enriched to a concentration of 1 mg/mL or higher.

This object is solved by the method of the present invention for assaying tropolone or a derivative thereof from animal cell culture supernatant or a proteinaceous solution containing an enriched product protein, comprising the steps of
a. separating the tropolone or its derivative from protein and, prior to that or after that step,
b. complexing the tropolone or its derivative with Cu(II)-ions in solution and
c. assaying tropolone or its derivative by means of reverse phase HPLC with a hydrophobic stationary phase and a mobile phase, preferably a water-miscible mobile phase, which mobile phase comprises comprises both Cu(II) ions and an ion-pairing reagent.

The figures exemplify a possible embodiment of the invention. What is shown is:

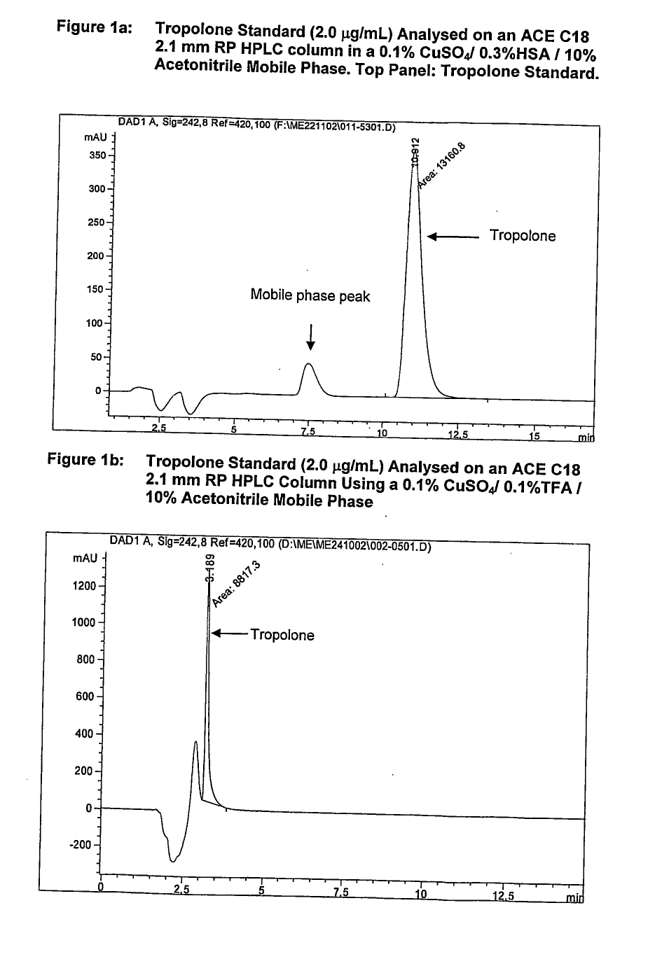

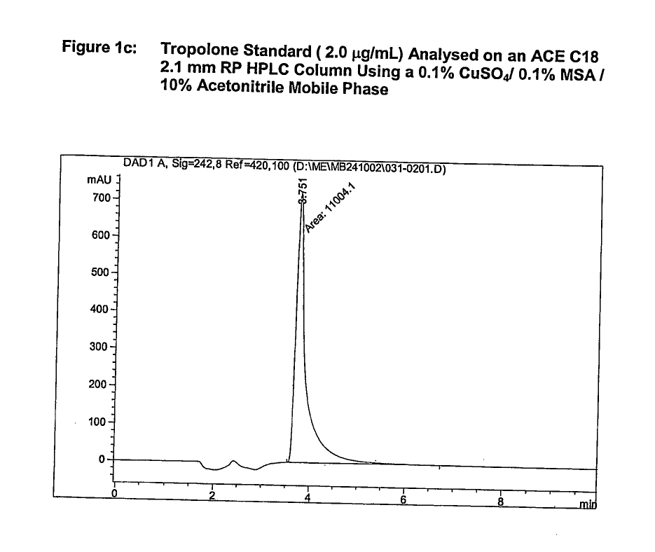

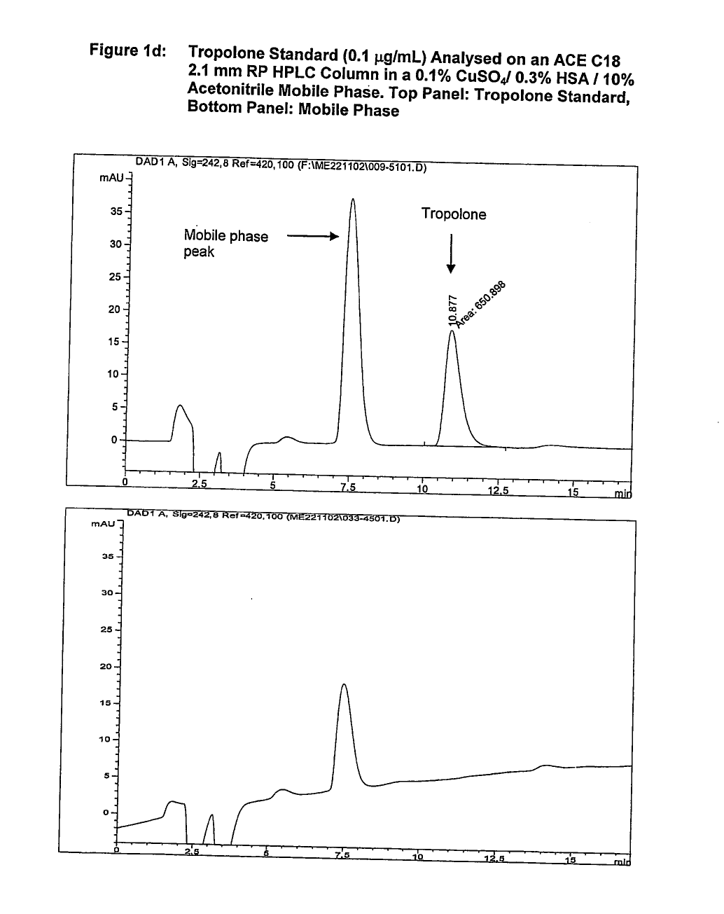

FIG. 1a: Tropolone Standard (2.0 μg/mL) Analysed on an ACE C18 2.1 mm RP HPLC column in a 0.1% CuSO₄/0.3% HSA/10% Acetonitrile Mobile Phase.

FIG. 1b: Tropolone Standard (2.0 μg/mL) Analysed on an ACE C18 2.1 mm RP HPLC Column Using a 0.1% CuSO₄/0.1% TFA/10% Acetonitrile Mobile Phase

FIG. 1c: Tropolone Standard (2.0 μg/mL) Analysed on an ACE C18 2.1 mm RP HPLC Column Using a 0.1% CuSO₄/0.1% MSA/10% Acetonitrile Mobile Phase

FIG. 1d: Tropolone Standard (0.1 μg/mL) Analysed on an ACE C18 2.1 mm RP HPLC Column in a 0.1% CuSO₄/0.3% HSA/10% Acetonitrile Mobile Phase. Top Panel: Tropolone Standard, Bottom Panel: Mobile Phase

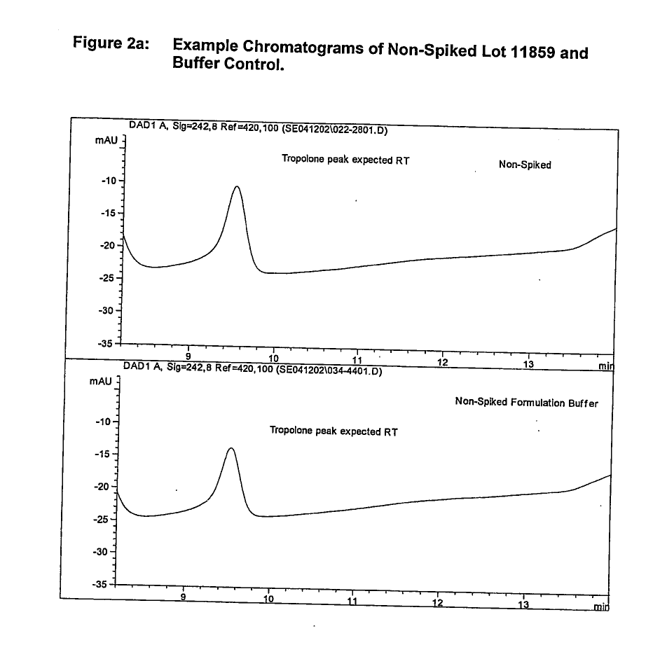

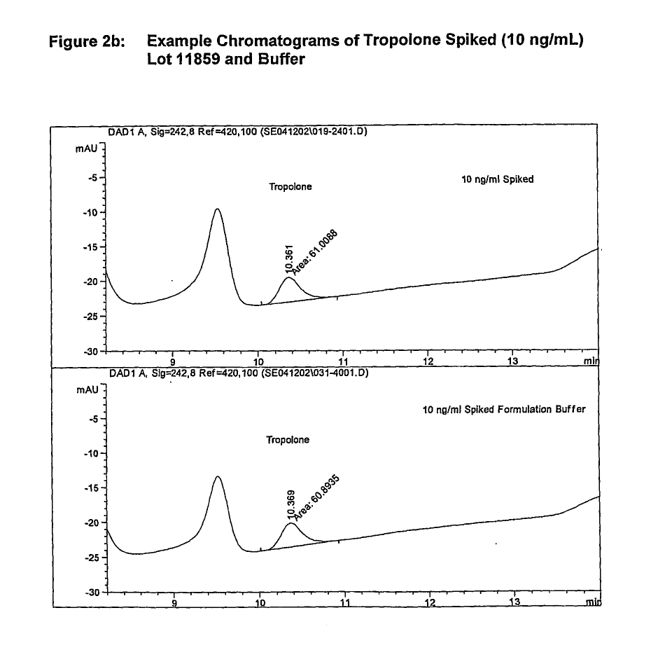

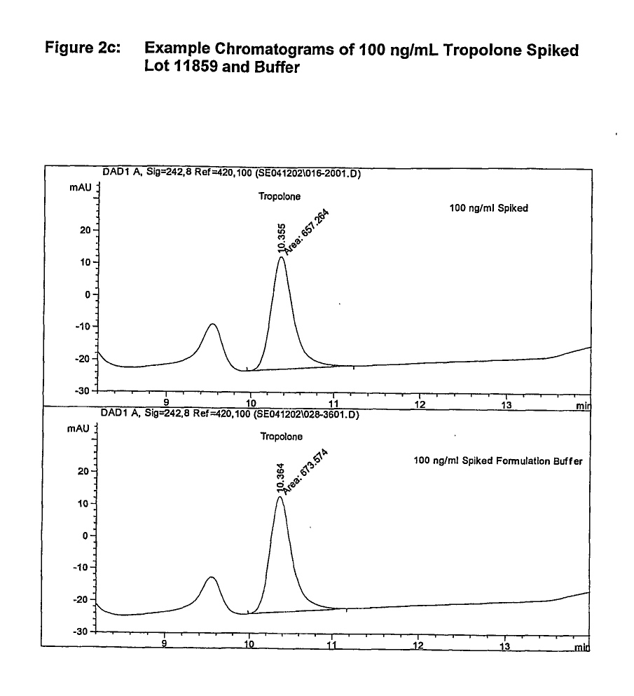

FIG. 2a: Example Chromatograms of Non-Spiked IgG Lot 11859 and Buffer Control.

FIG. 2b: Example Chromatograms of Tropolone Spiked (10 ng/mL) IgG Lot 11859 and spiked buffer control

FIG. 2c: Example Chromatograms of 100 ng/mL Tropolone Spiked IgG antibody Lot 11859 and spiked buffer control

Tropolone is 2-hydroxy-2,4,6-cycloheptatrienyl-1-one. Synthesis of Tropolone is described in Doering et al., J. Am. Chem. Soc. (1951), 73: 828-838. Synthesis of derivatives is described e.g. in U.S. Pat. No. 3,134,768 and JP44031595 B. According to the present invention, suitable derivatives of Tropolone are those derivatives which are capable of chelating ferrous or ferric ions and may be used as a membrane permeable, siderophoric iron chelator in animal cell culture without negatively affecting cellular function, i.e. without toxic side effects. Examples of a toxic tropolone derivative which is exempt from the scope of the present invention are e.g. colchicine or colchiceine which specifically disturb fundamental cellular functions and thus are highly toxic to cells; it is not possible to use or test them for siderophoric properties with living cells. Preferably, in order to allow of chelator properties, a suitable tropolone derivative according to the present invention complies with above definition and further has the structure I 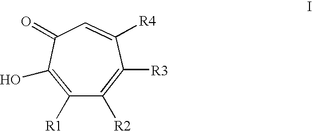
Wherein R1, R2, R3, R4 can be, alone or in combination, hydrogen, alkyl, alkenyl, alkynyl, aryl, aralkyl, alakenyl, alakynyl, heteroaryl, heteroaralkyl, heteroaralkenyl or heteroaralkynyl groups, with the proviso that R1═R2═R3═R4═H is excluded—which would be tropolone itself rather than a derivative thereof. The radicals R1 to R4 may be further substituted with e.g. halogeno, keto, amido functional groups where not interfering with the requirement of membrane permeability of a necessarily amphiphilic siderophore. ‘In combination’ means that radicals R1, R2, R3 and/or R4 may form cyclic substituents of the afore mentioned chemical types.

In a further preferred embodiment, it is Tropolone only that is assayed according to the method of the present invention. Derivatives of Tropolone are excluded in this way. Tropolone is very well known for its iron, but not for its Cu chelating properties. All preferred embodiments of the assay method and aspects of sample preparation described in this specification, where not relating explicitely to Tropolone derivatives, apply likewise to this preferred embodiment.

Reverse phase HPLC is well-known to the person skilled in the art and e.g. routinely applied in HPLC of peptides. In reverse phase HPLC, the stationary-phase consists of an inert support material having high mechanical strength such as typically e.g. a silica-based, coated aluminia-based or methacryl-polymeric support material. For reverse phase, a hyrophobic functional group is covalently bonded to the support or matrix material. Such reverse phase functional groups can be e.g. butyl, cyano, divinylphenyl, phenyl, hexyl, decyl, dodecacyl, octadecyl. It is also possible to use so-called shielded or embedded reverse phase columns of sufficient hydrophobicity and resolution properties, as is well-known to the skilled person. Suitable HPLC columns providing stationary phases for HPLC are readily commercially available, e.g. from Supelco/Fluka Chemie, Switzerland, or Waters/Mass., U.S.A.

Preferably, the reverse phase HPLC stationary phase is made up from a matrix functionalized with alkanyl, preferably with a C10 to C20 alkanyl, most preferably with C18-octadecylalkanyl. Further preferred is that that in conjunction with the preferred functional groups, the matrix material is silica. Thus the preferred stationary HPLC phase according to the present invention is an alkylsilane, more preferably a C10 to C20 alkylsilane, most preferably a C18-octadecyl-silane. These preferred embodiments are particularly advantageous in terms of resolution of tropolone peaks, column capacity and avoidance of sensitivity loss due to adsorption to the stationary phase when used in the method of the present invention along with the ion-pairing reagents and in particular its preferred embodiments described below.

An ion-pairing reagent according to the present invention is a reagent comprising at least one ionizable, carboxylic group that can at least transiently bind to the Cu-tropolone complex whilst having a certain affinity for the reverse phase stationary phase, thus retaining the Cu-tropolone complex on the stationary phase. In short, an ion-pairing reagent according to the present invention is defined by having a carboxylic group moiety and increasing the retention time of a Cu(II)-tropolone complex on a reverse phase-C18 HPLC column at 20° C. in 10% acetonitril in water in the presence of 0.1% (w/v) CuSO4.

Preferably, the ion-pairing reagent is more hydrophobic than trifluoroacetic acid (TFA). TFA is routinely employed in reverse phase HPLC of peptides and is almost considered a universal gold standard whence there is need for a ion pairing reagent for reverse phase HPLC. A more hydrophobic ion pairing compound is characterized, in its acid form, by a smaller numerical value of its dielectric material constant as compared to TFA. Suitable ion pairing reagents are e.g. tetrabutylammonium-phtalate, heptafluorobutyric acid, methylsulphonic acid, hexylsulphonic acid or the salts thereof. More preferably, the ion-pairing reagent has a hydrobobicity, as judged by the numercial value of its specific dielectric constant, that is equal to the hydrophobicity of or is in range between the hydrobobicity of methylsulphonic acid and hexylsulphonic acid. Most preferably, the ion pairing reagent is selected from the group consisting of butyl-sulphonic acid, pentyl-sulphonic acid and hexyl-sulphonic acid or a salt thereof, wherein the alkyl substituents may be branched or unbranched, preferably unbranched. In a particular preferred embodiment, the ion paring reagent is hexyl-sulphonic acid, more preferably n-hexyl-sulphonic acid, most preferably employed in a concentration of from 0.1 to 5% (w/v) in the mobile phase.

It may be possible, for the purposes of the present invention, to define hydrophobic properties for the purposes of reverse phase chromatography even more accurately by means of creating a retention index ranking for a given reverse phase HPLC embodiment of the present invention, using TFA, methylsulphonic and hexylsulphonic acid as the reference materials. Such index system has been e.g. used to define polarity of solvents (polarity index, L. R. Snyder, J. Chromatogr., 92, 223 (1974), J. Chromatogr. Sci., 16, 223 (1978) and may be adopted in a suitable context to define polarity/hydrophobicity of ion-pairing reagents).

Preferably, the concentration of the ion-pairing reagent is of from 0.01 to 10% (w/v), more preferably of from 0.1 to 5% (w/v), most preferably of from 0.11 to 1% (w/v). In a particular preferred embodiment, hexylsulphonic acid is used in a concentration of from 0.1 to 0.5% (w/v). In a further particularly preferred embodiment, hexalsulphonic acid in the contrations ranges described in this paragraph above is used in combination with an alkyl-silane stationary phase, preferably a C18-octadecyl-stationary-phase.

The mobile phase for HPLC expediently is a polar, water-miscible liquid. Preferably, the mobile phase comprises 1 to 30% (v/v), more preferably 5 to 20% (v/v) of acetonitrile. The acetonitrile is in admixture with at least one further polar solvent. The mobile phase may still comprise further solvents beside the further polar solvent and acetonitril.

Preferably, the further polar solvent is water, more preferably the mobile phase comprises at least 60% (v/v) water.

Preferably, the mobile phase comprises CuSO4 as a source of Cu(II)ions, in order to promote formation of the Cu(II)-tropolone complex and to avoid dissociation of the reversible complex upon HPLC. More preferably, the concentration of CuSO4 or the Cu(II) ions in general in the mobile phase is in the range of 0.05% to 0.2% (w/v), most preferably it is of from 0.7 to 0.14% (w/v).

As said above, it is preferred to assay Tropolone or its derivative from proteinaceous solution. More preferably from proteinaceous solution comprising a product protein that is enriched to a concentration of 1 mg/L or higher.

In a further preferred embodiment, the separation from protein is achieved by precipitating tropolone or its derivative along with protein with a Cu(II)-salt, preferably with CuSO4, and recovering the precipitate and secondly removing protein from the recovered precipitate by ultrafiltration. Ultrafiltration may be carried out with devices well-known to the protein or biochemist, ie. Amicon filter membranes or spin filters using such membranes. This preferred embodiment allows of very efficient, complete recovery of tropolone or its derivative from the original sample whilst allowing of complex formation in a single step along with separation. Most importantly, the method allows of eliminating interfering ionic species (phosphate, chloride) that are usually present in buffers used for storing non-denatured protein and which ionic species would interfer with the HPLC assay for tropolone. No desalting of sample is required for operation of HPLC. Further, there is no need for desalting prior to loading of the sample onto the HPLC. In contrast ammonium sulfate precipitation as commonly employed for protein precipitation in the art would, however, result in incomplete recovery of tropolone. Expediently, precipitation is carried out with 0.5 to 10% Cu(II)-salt, more preferably with 1 to 5% (w/v) Cu(II) salt.

### Experiments

Different routes were tested for sample preparation from purified, complete IgG antibody solution made up from standard PBS buffer (physiological phosphate buffered saline) and spiked with 2000 ng/mL of tropolone. A cell culture medium usually comprises around 10 μM of tropolone from the onset of cell culture.

By comparing different methods of working up identical samples, the method resulting in greatest yield of tropolone from the original sample was determined. The best working, precipitation with Cu(II), is described in more detail.

The method was found to perform equally well with true in-process samples derived from crudely purified IgG antibody from NS0 cell culture supernatant which cell culture was grown in the presence of 30 μM tropolone. The supernatant had only been processed with Protein A-Sepharose chromatography, both spiked and unspiked samples were tested.

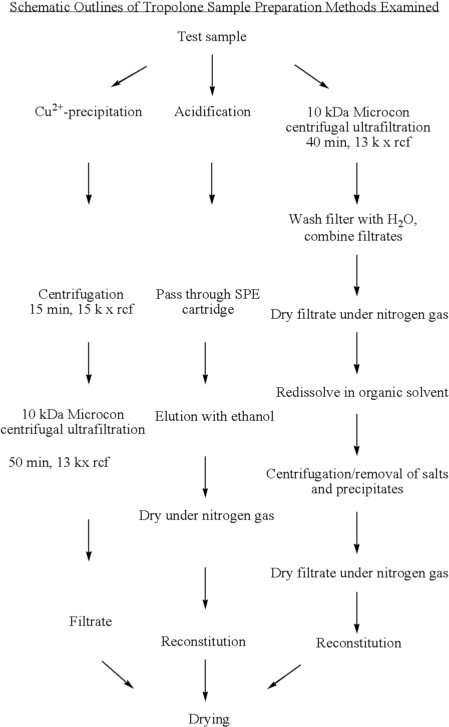

After drying in the Ultravap nitrogen drying unit, worked-up samples were reconstituted in 200 μL mobile phase for analysis by RP HPLC as described above. SPE: gravity-operated Strata X 3 mL solid phase extraction cartridges made up from a polystyrene divinyl benzene solid polymer, pre-conditioned with 500 μL ethanol and 500 μL 5 mM H2SO4.

1. CuSO₄ Sample Precipitation

Standard tropolone solutions prepared as said above were mixed with CuSO₄ solution to a final concentration of 2% CuSO₄ (w/v). Precipitated salts were removed by centrifugation at 15 000×rcf for 15 minutes.

Microcon® (Millipore, USA) 10 kDA MW-cut off 500 μL-filters (YM-10) were equilibrated by the addition of 400 μL H₂O followed by centrifugation for 40 minutes at 13 000×rcf. The sample supernatants were then applied to the Microcon filters. The filters were centrifuged for 40 minutes at 13 000×rcf. The filters were then washed with 100 μL H₂O and the filtrates pooled. The pooled filtrates were dried in glass vials under nitrogen, reconstituted in 200 μL mobile phase and analysed by RP HPLC. The method resulted in a 84% yield. In general, for samples spiked with 100 to 2000 ng/mL tropolone this mean recovery proved reproducible; for samples spiked with very low amounts of tropolone in the 20-40 ng/mL range, recovery rates were as high as 94%.

Alternatively, this procedure was performed without drying of samples; drying proved not to be required in the optimized procedure. The filtrates were analysed neat by RP HPLC.

The optional work-up methods shortly described above resulted in mean recovery rates of <20% (solid phase extraction) and 65% (Microcon ultrafiltration).

2. RP HPLC

RP HPLC was carried out with an Advanced Chromatography Technologies ACE C18 2.1 mm column as the stationary phase and a 0.1% CuSO4 (w/v)/0.3% hexane sulphonic acid/10% acetonitrile-in-water mobile phase, under conditions of isocratic elution, a flow rate 0.2 mL/min at 25° C., detection at 242 nm and 100 μL injection volume. The HPLC system was an Agilent 1100 HPLC system or equivlanet, comprising vacuum degasser, binary pump, thermostatted column compartment and autosampler, along with Chemstation software version A6.04 or higher.

The elution curve for a tropolone standard analyzed in this way is shown in FIGS. 1a and 1d. FIGS. 1b and 1 c show elution curves with the same mobile and stationary phase, except that different ion pairing reagents were used (TFA and MSA instead, s. section 4).

FIG. 2a-2b show analysis of an in process-lot of complete IgG antibody derived from large-scale production. The antibody was only partially purified by means of Protein A-Sepharose chromatography. Further explanations are given in the figures' captions.

A comparison of the ACE C-18 column with a Hamilton [company location?] PRP Polystyrene-Divinylbenzene 4.6 mm reverse phase column was carried out. The PRP-1 polymeric column evaluated previously was re-investigated for use with CuSO₄ mobile phases as the tolerance of the polystyrene-divinylbenzene stationary phase for aqueous solutions permits the use of higher polarity mobile phases (compared to C18 columns) to potentially increase the retention time of tropolone elution. As tropolone failed to elute with 0.1% CuSO₄ mobile phase after 15 minutes in the absence of acetonitrile (data not shown), varying percentages acetonitrile (isocratic 5% (followed by organic column clean-up step), 21%, 40% and 3% to 11.5% gradient) were evaluated. The flow rate used was constant at 0.8 mL/min at a temperature of 25° C.

Mobile phases were then evaluated with various acetonitrile contents and the results from analyses under these conditions are summarised in Table 3. At a concentration of 5% acetonitrile, acceptable retention (k>1.0), tolerable tailing (TF 3.02) and reproducible detection of 0.1 μg/mL tropolone standard was achieved. However, the normalised peak height of 2 indicated 0.1 μg/mL was close to the limit of detection using these conditions (Table 3). In addition, peak tailing at this concentration was much higher than for 2.0 μg/mL (TF 1.61). At higher concentrations of acetonitrile (21% and 40%), retention of tropolone was k<0.5, and thus unacceptable.

TABLE 3
Tropolone Standard (0.1 μg/mL and 2.0 μg/mL) Analysis by RP HPLC on Three Columns Using
Different Mobile Phases Containing 0.1% CuSO₄

| | | | | | | 2.0 μg/mL | | | | | 0.1 μg/mL | | | | |
|---|---|---|---|---|---|---|---|---|---|---|---|---|---|---|---|
| Column/ | % Acid/ | | | | | | % CV | | | | Normalised | % CV | | | |
| ID | IP | % | % | T | Flow rate | Normalised | Peak | | | | Peak | Peak | | | |
| (mm)¹ | Reagent | CuSO₄ | CH₃CN | (° C.) | (mL/min) | Peak Height⁵ | Area | TF² | k³ | Rₛ⁴ | Height⁵ | Area | TF² | K³ | Rₛ⁴ |
| ACE | NT | 0.1 | 10 | 25 | 0.6 | 476 | NT | 1.20 | 0.37 | N/A | 13 | NT | 1.92 | 0.36 | N/A |
| C18/4.6 | | | | | | | | | | | | | | | |
| ACE | NT | 0.1 | 5 | 25 | 0.2 | 2069 | 0.7 | 11.28 | 0.57 | N/A | NT | N/A | N/A | N/A | N/A |
| C18/2.1 | | | | | | | | | | | | | | | |
| ACE | NT | 0.1 | 20 | 25 | 0.2 | 437 | 3.2 | 1.91 | 0.28 | N/A | ND | N/A | N/A | N/A | N/A |
| C18/2.1 | | | | | | | | | | | | | | | |
| ACE | NT | 0.1 | 50 | 25 | 0.2 | 532 | 1.7 | 2.39 | 0.0 | N/A | NT | N/A | N/A | N/A | N/A |
| C18/2.1 | | | | | | | | | | | | | | | |
| PRP-1/4.6 | NT | 0.1 | 5 | 25 | 0.8 | 114 | NT | 1.61 | 1.34 | N/A | 2 | NT | 3.02 | 1.30 | N/A |
| PRP-1/4.6 | NT | 0.1 | 21 | 25 | 0.8 | 972 | NT | 1.25 | 0.29 | N/A | ND | N/A | N/A | N/A | N/A |
| PRP-1/4.6 | NT | 0.1 | 40 | 25 | 0.8 | 282 | 1.0 | 1.78 | 0.46 | N/A | 8 | 5.9 | 4.69 | 0.48 | N/A |
| PRP-1/4.6 | NT | 0.1 | 3-11.5 | 25 | 0.8 | 104 | NT | 1.19 | 1.98 | N/A | NT | N/A | N/A | N/A | N/A |

Detection by UV absorbance at 242 nm (420 nm reference) and 100 μL Injection volume.
¹Columns: Symmetry C18: Waters Symmetry C18; ACE C18: Advanced Chromatography Technology ACE C18; PRP-1: Hamilton PRP-1 PSDVB polymer
²TF: USP Tailing factor.
³K: Retention factor.
⁴Rs: USP Resolution factor.
⁵Normalised Peak Height: Response on Sym C18 column at 2.0 μg/mL = 100% (Absolute peak height of 70.3 mAU * S).
ND: Not Detected;
NT: Not Tested;
N/A: Not Applicable.

3. HPLC Performance

All studies below were using the RP HPLC method of experiment 2 above and sample preparation method of experiment 1 above.

(a) Linearity

Tropolone solutions were prepared in antibody comprising buffer as set forth above at concentrations of 0, 1, 5, 10, 20, 50, 100, 500, 2000 and 4000 ng/mL tropolone. Buffer salt removal was carried out by precipitation with 2% CuSO₄. The tropolone samples were analysed by RP HPLC. Peak areas were recorded and plotted against tropolone concentration and linear regression plots were constructed. The linear working range was defined as the concentration range giving a linear response (r²≧0.99) with acceptable precision (≦5% CV). Calibration curves for tropolone quantitation were constructed from samples analysed at concentrations of 5, 10, 20, 50, 100, 500, 2000 and 4000 ng/mL tropolone. The precision of measurement of each tropolone concentration was analysed. A linear response was observed both visually between 1 ng/mL and 4000 ng/mL and by regression analysis. The square of the correlation coefficient (r²) value indicated acceptable linearity (r²=0.9997).

For routine quantitation purposes, calibration curves were constructed for concentrations between 0 ng/mL and 2000 ng/mL (r²>0.99). Linearity was examined on five different occasions and the regression data are summarised in Table 1.

TABLE 1
Regression Data from Five Assays

| Assay reference | r² | Slope | Intercept | P (intercept) |
|---|---|---|---|---|
| Final evaluation | 0.9999 | 6.1244 | −21.70 | 0.14 |
| Accuracy 1 | 1.0000 | 6.0353 | −2.02 | 0.67 |
| Accuracy 2 | 1.0000 | 6.7327 | −10.68 | 0.05 |
| Accuracy 3 | 1.0000 | 6.3212 | −11.04 | 0.07 |
| In-process | 1.0000 | 6.4452 | −6.14 | 0.34 |

(b) Specificity

Resolution (Rₛ) of the tropolone peak from sample buffer related peaks was determined for standard tropolone solutions (2000 ng/mL). The mean Rs, sd and % CV were recorded over five assays from triplicate analyses.

As discussed previously, a sample buffer related peak which was visually resolved from tropolone eluted immediately before tropolone (Section 4.5.2; FIGS. 18 and 19). The resolution of tropolone from this buffer-related peak was calculated for three system suitability sample injections in five separate assays using the USP defined method. The mean troplone resolution factor (Rs), sd and % CV were calculated and the results summarised in Table 2.

TABLE 2
Resolution of Tropolone System Suitability Samples (2000 ng/mL)
from the Formulation Buffer Related Peak.

| Assay | Mean Rs | sd | % CV |
|---|---|---|---|
| Accuracy 1 | N/A | N/A | N/A |
| Accuracy 2 | 1.7 | 0.0 | 0.8 |
| Accuracy 3 | 1.6 | 0.0 | 1.6 |
| In-process | 1.5 | 0.0 | 1.4 |
| Lot 17362 | 1.5 | 0.0 | 1.6 |

N/A: Not applicable as no formulation buffer peak detected.

The mean resolution factors were all ≧1.5, indicating acceptable resolution from the preceding buffer related peak and demonstrated acceptable repeatability (CV<3.0%). No interfering peaks were observed from sample preparation or final product samples. This confirmed the specificity of the RP HPLC assay for the detection and quantitation of tropolone.

(c) Limit of Detection/Quantitation (LOD/LOQ)

Tropolone samples analysed for linearity were used to estimate the LOD and LOQ for tropolone analysis. The lowest concentration of tropolone tested giving a detectable signal was assigned as the LOD. The lowest concentration of tropolone which could be measured with acceptable repeatability (≦5% CV) was defined as the LOQ. This was found to amound to LOD approx. 1 ng/mL; the LOQ was determined as about 5 ng/mL, and thus the sensitivity limit for the assay method of the present invention.

4. Evaluation of HPLC Conditions on the ACE C18 2.1 mm Column Using Copper-Ion Complexation with Ion Pairing (IP) Reagents

Analysis of the copper-ion tropolone complex on C18 HPLC columns were tested with reversed phase ion pairing using three different reagents of varying hydrophobicity. These were trifluoroacetic acid (TFA), the most polar, methylsulphonic acid (MSA), moderately polar and hexanesulphonic acid (HSA), the most hydrophobic. Mobile phases were prepared containing the ion pairing reagent, 0.1% CuSO₄ and acetonitrile. HPLC parameters used are summarised in Table 4 together with the obtained relative peak heights and chromatographic test measures.

4.1 TFA Ion Pairing

Retention of tropolone standards was not sufficiently increased (k<1.0) using 0.1% TFA in the mobile phase to be acceptable (Table 4, FIG. 10). Analysis under these conditions was concluded to be not suitable for assay of tropolone.

4.2 MSA Ion Pairing

Mobile phase containing 0.1% MSA gave improved retention time (k≧2) under isocratic elution with 5% acetonitrile and 0.1% MSA. However, high tropolone peak tailing was observed (TF≧3.4) for both concentrations of standard tested (0.1 μg/mL and 2.0 μg/mL). The use of mobile phase containing 0.1% MSA and 10% acetonitrile (FIG. 11) demonstrated improved detection of the 0.1 μg/mL tropolone standard (normalised peak height of 14; Table 4) than for 5% acetonitrile/0.1% MSA (normalised peak height of 2), but peak tailing was extended (TF 12.05).

4.3 HSA Ion Pairing

A significant increase in tropolone retention time was achieved using isocratic elution with a 0.1% HSA/10% acetonitrile mobile phase (k≧3.4; Table 4). Furthermore, increased peak heights for low tropolone concentrations of 0.1 μg/mL were observed (normalised peak height of 27; Table 4) and peak tailing was acceptable and comparable for both tropolone concentrations tested (TF≦1.9). Furthermore, peak area repeatability was acceptable at concentrations of 2.0 μg/mL (0.9% CV) and 0.1 μg/mL (3.2% CV) tropolone (Table 4). However, tropolone eluted close to a mobile phase peak and the two peaks did not demonstrate baseline resolution (FIGS. 12 and 13; Rₛ≦1.1). The tropolone and mobile phase peaks were not able to be separated using a gradient of acetonitrile (10% to 42%) nor by increasing the analysis temperature to 40° C. (Rs≦1.0, Table 4) or decreasing the temperature to 20° C. and the flow rate to 0.1 mL/min (Rs≦1.3).

Variation of the ion-pair (HSA) mobile phase concentration was evaluated. A 0.3% HSA/10% acetonitrile/0.1% CuSO₄ mobile phase (flow 0.2 mL/min, 25° C.) was effective for achieving baseline separation of tropolone from the mobile phase peak at both tropolone concentrations (Rₛ≧3.5, FIGS. 14 and 15, Table 4). A further decrease in flow rate (0.1 mL/min) did not lead to any further improvement in tropolone resolution (Rs≧3.8). In conclusion, a RP HPLC method was established with acceptable limits of detection, peak symmetry and reproducibility for the assay of tropolone standards.

TABLE 4
Tropolone Standard (0.1 μg/mL and 2.0 μg/mL) Analysis on the ACE C18 RP HPLC Using
Mobile Phases Containing 0.1% CuSO₄ and Three Different Ion Pairing Reagents.

| | | | | | Flow | 2.0 μg/mL | | | | | 0.1 μg/mL | | | | |
|---|---|---|---|---|---|---|---|---|---|---|---|---|---|---|---|
| Column/ | % Acid/ | | | | rate | Normalised | % CV | | | | Normalised | % CV | | | |
| ID | IP | % | % | T | (mL/ | Peak | Peak | | | | Peak | Peak | | | |
| (mm)¹ | Reagent | CuSO₄ | CH₃CN | (° C.) | min) | Height⁵ | Area | TF² | k³ | Rs⁴ | Height⁵ | Area | TF² | k³ | Rs⁴ |
| ACE | 0.1/TFA | 0.1 | 5 | 25 | 0.2 | 1842 | NT | 2.21 | 0.75 | N/A | NT | N/A | N/A | N/A | N/A |
| C18/2.1 | | | | | | | | | | | | | | | |
| ACE | 0.1/MSA | 0.1 | 5 | 25 | 0.2 | 348 | NT | 3.44 | 2.00 | N/A | 2⁶ | NT | 9.64 | 2.06 | N/A |
| C18/2.1 | | | | | | | | | | | | | | | |
| ACE | 0.1/MSA | 0.1 | 10 | 25 | 0.2 | 1074 | 3.6 | 2.68 | 1.06 | N/A | 14⁶ | 0.5 | 12.05 | 0.83 | N/A |
| C18/2.1 | | | | | | | | | | | | | | | |
| ACE | 0.1/HSA | 0.1 | 10 | 25 | 0.2 | 526 | 0.9 | 1.62 | 3.48 | 1.1 | 27 | 3.2 | 1.87 | 3.46 | 0.8 |
| C18/2.1 | | | | | | | | | | | | | | | |
| ACE | 0.1/HSA | 0.1 | 10-42 | 25 | 0.2 | 601 | 0.3 | 1.54 | 3.42 | 1.0 | 32 | 1.1 | 1.51 | 3.40 | 0.8 |
| C18/2.1 | | | | | | | | | | | | | | | |
| ACE | 0.1/HSA | 0.1 | 10 | 40 | 0.2 | 598 | 1.0 | 1.61 | 3.30 | 0.9 | NT | N/A | N/A | N/A | N/A |
| C18/2.1 | | | | | | | | | | | | | | | |
| ACE | 0.1/HSA | 0.1 | 10 | 20 | 0.1 | 613 | 0.9 | 1.50 | 3.80 | 1.3 | 31 | 0.1 | 1.74 | 3.79 | 1.2 |
| C18/2.1 | | | | | | | | | | | | | | | |
| ACE | 0.3/HSA | 0.1 | 10 | 25 | 0.2 | 493 | 0.4 | 1.60 | 4.81 | 3.6 | 23 | 9.1 | 1.65 | 4.80 | 3.7 |
| C18/2.1 | | | | | | | | | | | | | | | |
| ACE | 0.3/HSA | 0.1 | 10 | 20 | 0.1 | 535 | 0.6 | 1.40 | 4.91 | 4.2 | 24 | 3.2 | 1.71 | 4.92 | 3.8 |
| C18/2.1 | | | | | | | | | | | | | | | |

Detection by UV absorbance at 242 nm (420 nm reference) and 100 μL Injection volume.
¹Colunms: Symmetry C18: Waters Symmetry C18; ACE C18: Advanced Chromatography Technology ACE C18; PRP-1: Hamilton PRP-1 PSDVB polymer
²TF: USP Tailing factor.
³K: Retention factor.
⁴Rs: USP Resolution factor.
⁵Normalised Peak Height: Response on Sym C18 column at 2.0 μg/mL = 100% (Absolute peak height of 70.3 mAU).
⁶5 μL Injection Volume.
ND: Not Detected;
NT: Not Tested;
N/A: Not Applicable.

1-13. (canceled)

14. A method for assaying tropolone or a derivative thereof of structure I 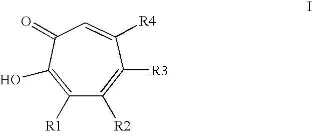
wherein R1, R2, R3, R4 can be, alone or in combination, hydrogen, alkyl, alkenyl, alkynyl, aryl, aralkyl, aralkenyl, aralkynyl, heteroaryl, heteroaralkyl, heteroaralkenyl or heteroaralkynyl groups, from animal cell culture supernatant or a proteinaceous solution containing an enriched product protein, comprising the steps of
a. Separating the tropolone or its derivative from protein and, prior to that step or after that step, complexing the tropolone or its derivative with Cu(II)-ions in solution
b. Assaying tropolone or its derivative by means of reverse phase HPLC with a hydrophobic stationary phase and a mobile phase which mobile phase comprises both Cu(II) ions and an ion-pairing reagent, characterized in that the ion-pairing reagent is an ion-pairing carboxylic acid, or a salt thereof, that is more hydrophobic than trifluoroacetic acid.

15. Method according to claim 14, characterised in that the hydrophobic stationary phase is an alkyl-silane stationary phase.

16. Method according to claim 15, characterised in that the alkyl-silane stationary phase is an unbranched alkyl-silane phase, preferably is a C-18 alkyl-silane.

17. Method according to claim 14, characterised in that the ion-pairing reagent has a dielectric constant that is equal to the dielectric constants of methylsulphonic acid or hexylsulphonic acid or is in the range defined by the dielectric constants of methylsulphonic acid and hexylsulphonic acid.

18. Method according to claim 17, characterised in that the ion-pairing reagent is selected from the group consisting of propyl-sulphonic acid, butylsulphonic acid, pentylsulphonic acid, hexylsulphonic acid and salts of thereof

19. Method according to claim 14, characterised in that the mobile phase comprises 1 to 30% of acetonitrile in admixture with at least one further polar solvent.

20. Method according to claim 19, characterised in the the polar solvent is water, methanol, ethanol or an admixture thereof, preferably the mobile phase comprises at least 60% water.

21. Method according to claim 14, characterised in that the separation from protein is achieved by firstly precipitating tropolone or its derivative with CuSO₄ and recovering the precipitate and secondly removing protein from the recovered precipitate by ultrafiltration.

22. Method according to claim 14, characterized in that the mobile phase comprises CuSO4 as a source of Cu(II) ions.

23. Method according to claim 22, characterized in that the concentration of CuSO₄ in the mobile phase is in the range of 0.05% (w/v) to 0.2% (w/v).

24. Method according to claim 14, characterised in the mobile phase is watermiscible.

25. Method according to claim 14, characterised in that the supernatant or proteinaceous solution comprising protein is enriched to a concentration of 1 mg/ml or higher.
